(19) United States
(12) Patent Application Publication
(10) Pub. No.: US 2020/0357512 A1
(43) Pub. Date: Nov. 12, 2020

(54) UNIFIED PROCESS FOR TRANSMISSIBLE VIRAL PATHOGENS RADIAL PROXIMITY REAL-TIME VIRTUAL GLOBAL POSITIONING PLOTTING AND SOCIAL DISTANCING COMPUTERIZED ADVANCED ADVISORY REGISTRY DRIVEN PLATFORM MEDICAL SERVICE

(71) Applicant: ROBERT GEORGE COGHLAN, Hialeah, FL (US)

(72) Inventor: ROBERT GEORGE COGHLAN, Hialeah, FL (US)

(21) Appl. No.: 16/941,087

(22) Filed: Jul. 28, 2020

Related U.S. Application Data
(63) Continuation of application No. 16/914,433, filed on Jun. 28, 2020.

(51) Int. Cl. G16H 40/60; G16H 10/60; G16H 70/60

(52) U.S. Cl. CPC G16H 40/60; G16H 70/60; G16H 10/60

(57) ABSTRACT

Unified process for transmissible viral pathogen radial proximity real-time virtual global positioning plotting and social distancing computerized advanced advisory registry driven platform medical service in the field of endeavor in the technological arts of providing a means to mitigate a transmission of a viral pathogen, such as though not limited to, Covid19, and providing a registry and a platform medical service for advising a primary individual of secondary individuals in a radial proximity to the primary individual of a health status of a secondary individual within a radial proximity of the primary individual by the means of a real-time virtual global positioning plotting and social distancing computerized advanced advisory registry driven platform medical service.

### BACKGROUND OF THE INVENTION

### 1. Field of the Invention

The present invention relates to the field of endeavor of a processes for providing a registry and a platform medical service for advising a primary individual of secondary individuals in a radial proximity to the primary individual of a health status of a secondary individual within a radial proximity of the primary individual. More particularly, the present invention provides a unified process for transmissible viral pathogens radial proximity real-time virtual global positioning plotting and social distancing computerized advanced advisory registry driven platform medical service.

### 2. Description of Prior Art and Background

The current state of the art in the field of endeavor of a unified process for transmissible viral pathogens radial proximity real-time virtual global positioning plotting and social distancing computerized advisory registry driven platform medical service exhibits problems. Such exhibited problems involve computerized applications where an “App”, through utilizing a “Bluetooth”™ application on a computerized device of an individual measures ‘close contacts’ between individuals and issues a warning to a primary individual, “later” when a secondary individual tests positive for a viral pathogen. As such, current technology in the form of currently practice social distancing and contact tracing apps that are freely pooled on the World Wide Web a require no medically material pre-condition, such as a health status of an individual as determined by a medical professional in order to practice such apps, bear limited to no near real-time means to advance a registry driven platform populated by a medical professional and an associated map plotting computerized monitor display that “forewarns” a primary individual of a secondary individual within a radial proximity as determined by a global positioning data that a secondary individual is in good or not good health, and advising of a primary individual to follow a protocol and be responsive and avoid an encounter within a preset radial distance of a secondary individual who has been determined to not be in good health by a medical professional that may potentially violate a primary individual's social distancing practice and subsequently, through a process of personal and/or surface touching or airborne transmissibility, become infected with a viral pathogen as initially carried and generated by a secondary individual.

Exercise of the contemporary practice of ‘contact tracing and social distancing apps’ make known such limited means as such apps provide solely for a unilateral information stream that is often provided during or after a social distancing relation between parties. For these matters, the prevailing state of the art of viral pathogen transmissibility technology, specifically in the form of real-time advanced advisory necessitates technological modernization in terms of a real-time advanced advisory bilateral information stream to a plurality of socially engaging individuals in order to attend to the long-felt need, and indeed urgent need, of securing the health and well-being of a population from a viral pathogen such as a viral pandemic, in the present instance, Covid19.

When an individual, such as a primary individual is at home, in a work facility, a worshiping place, an educational setting, a public park or other public settings such as a supermarket, such individual has limited means to assess the health of other individuals in radial proximity to the individual in view of the failure of current technology to provide for an additive level of information, such as a virtual map plotting on a computerized device's monitor of an individual in order to inform a primary individual of a health status of a secondary individual and a primary individual global positioning proximity to a secondary individual. As well, there are limited or no unified means to determine if and when a given public setting is free and clear or not free and clear for a primary individual to be active within as determined by a radial plotting virtual display on a computerized device of a primary individual and programmatically advise a primary individual of the health status of a secondary individual within a radial proximity to the primary individual in a given public or social setting.

Moreover, there no advanced advisory means that condition a primary individual to be responsive on the occasion when a primary individual may potentially encounter a secondary individual that has been determined by a Medical Professional to not be in good health, and therefore, expose a primary individual to a secondary individual, and whereas, the primary individual may, through the foregoing description of personal or surface touch or airborne transmissibility, acquire a viral pathogen from a secondary individual, such as Covid19, and unwittingly transmit the virus to a third individual.

In essence, current art bears no means to advance a unified process for transmissible viral pathogens radial proximity real-time virtual global positioning plotting and social distancing computerized advisory registry driven platform medical service in order to assist in a social effort to contain a viral pandemic such a Covid19. As well, present technological forms exhibited in the aforementioned ‘contact apps’ fail to provide a means to negate an interaction, such as a violation of a social distancing between a set of individuals, such as a primary individual and a secondary individual, and as a consequence of such failure, lack means to prevent transmissibility of a viral pathogen.

The present invention's medical service addresses such long-felt social, and indeed ‘urgent’ need, whereby it advances such a platform medical service as defined within the present invention's claims, specification, drawings and abstract, as it provides means for individuals, such as a primary and a secondary individual to be programmatically advised when the individuals come within a radial proximity as determined by a global positioning and take preventative measures, such as maintaining a social distance that follows a protocol in order to thwart a transmission of a viral pathogen.

Worldwide viral pathogens, specifically viral pathogen cases involving a viral pathogen named, “Covid19”, increased dramatically during the 2019-2020 calendar year. As a result, the “World Health Organization” (WHO), Director-General Dr Tedros Adhanom Ghebreyesus declared the 2019-nCoV outbreak a Public Health Emergency of International Concern on 11 Mar. 2020, following a second meeting of the Emergency Committee convened under the International Health Regulations. Countries around the world were ill prepared to provide health care to infected individuals, and as well contain the spread of the viral pathogen, Covid19. Hence, as there was a critical shortage, ‘understaffing’, of medical personnel and medical equipment to provide health care to infected individuals, and as a consequence, there continues to be sickness, mortality and widespread economic hardships placed upon persons worldwide. Such understaffing, and lack of medical personnel and medical equipment led to government agencies worldwide taking abrupt measures such as “lockdowns” where everyone practices social distancing from everyone and in turn, negatively exacerbates the physical, psychological, emotional and economic characteristic of persons under such lockdown methods. As persons who do not currently have access to the process of the present invention's medical service such person singular recourse is to socially isolate from as many, including all persons, as possible as such person is unaware of the health status of other individuals. When a person is without the processes of the present invention's medical service and has not been tested and assigned a health status, such person is understood to be, “unassigned”. Presently, all persons globally are understood to be unassigned in relation to the processes of the present invention's medical service. In such view, persons tend to “err on the side of caution” when encountering a secondary individual and practice a social distancing technique, even when the practice of a social distance may not be required. As a result, individuals become increasingly social isolated and often experiences adverse contra-indications from social isolation such as the foregoing descriptions of physical, psychological and emotional well-being.

The present invention's medical service is drawn toward the interests of providing resolution to such problem in its field of endeavor as the state of technology in the art of advanced advisory of potential viral pathogen transmissibility will be materially enhanced by the present invention's inventive leap of providing a registry driven platform medical service so individuals may be assigned a health status by a medical professional, utilize the process of the present invention's medical service and carry on with their life's general affairs in a reasonable and more confident manner as such invention will provide means to inform primary individuals of secondary individuals that are in a radial proximity of the primary individual and who are determined to be not in good health, and subsequently both parties exercise a social distance, and as a consequence, assist in the containment of a viral pandemic.

Social distancing applications provide means for persons to know when they are within a given proximity to one another. In some instances, though not all, a distance is qualified as one party may be carrying a viral pathogen that could infect another party. However, social distancing applications often require that persons establish a social distance when a social distance is not required. Therefore, a social distancing application unnecessarily exacerbates a social isolation of a person who may otherwise freely engage in social activity if the person had a higher level of knowledge about a person or persons that such person wished to socially engage with. For example, a person wishing to patronize a supermarket may, by the means of a social distancing app, maintain a social distance from all persons the person may encounter and hence, not freely engage in a social activity, such as a conversation at a supermarket. A condition of our specie, primates, homo-sapiens, is to be socially interactive when an interest in social interaction occurs to an individual. For such matter, while a main objective of patronizing a supermarket of purchasing goods may be in the forefront of an individual's thoughts, a corollary benefit of patronizing a supermarket is one that provides an individual with means to be socially interactive in terms of exchanging pleasantries and short conversation with persons an individual meets along the way of a visit to a supermarket. A social distancing app often quells an interest, even an ambition of an individual to be socially interactive, as such individual cannot decipher who is in good health and available for social interaction, such as conversation, and who is not in good health and not available for social interaction, such as conversation. As a consequence, persons utilizing a social distancing app often engage in conversation at a social distance and for a limited time frame, or do not engage in social interaction, such as conversation, and patronize a supermarket for the sheer utility of purchasing goods. A problem becomes apparent, and as well highlighted, when such social distancing application limits or prevents an individual from social interactivity. The problem is that of social isolation as further exacerbated by the failure of social distancing applications to provide a level of information to an individual so such individual may exercise a condition of our specie and socially interact.

The present invention medical service, addresses and in fact ‘establishes’ a level of information about persons an individual, such as an individual who is patronizing a supermarket for the purposes of purchasing goods and as well entertaining a social interaction such as a conversation by its means to provide an information medical service to a level and degree not currently in practice such as knowing who such individual should engage socially and who such individual should not engage socially. Effectually, the present invention's medical service provides a social “filter” as its processes provide means to distinguish when a person is in good health and available for social interaction and when a person is not in good health and not available for social interaction. As such, and as expressed in the present inventions claims, as, “programming the central computer to determine a global position of a primary and secondary MEDIHS and “only” when a radial distance of a primary MEDIHS is being breached by a secondary MEDIHS the central computer programmatically transmits an advanced advisory to a primary and secondary MEDIHS of primary IHS and secondary IHS in real-time” the present invention transmits an advanced advisory—“only” when a radial distance of a primary IHS is being breeched by a secondary IHS with a health status as, “Not good”. The present invention, by these means, provides for a more confident and secure mindset of an individual to be socially interactive as desired on an occasion such as a visit to a supermarket.

While a person who has been tested as, “positive”, for a viral pathogen, such as Covid19, and is practicing a self-quarantine and socially isolating, it should be understood that is not always that a person, even willfully, may remain in a self-quarantine. Sometimes, circumstances arise that warrant a person who is self-quarantining to become socially interactive. For example, an individual who is self-quarantining and in social isolation in light of a positive test for a viral pathogen, such as Covid19, may experience a health issue, such as cardiac-arrest and need to be transported by a medic to a medical facility. When such person is experiencing a state of cardiac-arrest, such person is often in a state of impairment of mental cognition and unable to inform a medic of a health consideration such as an infection of a viral pathogen such as Covid19, subsequently and unintentionally exposing attending medics and physicians to the viral pathogen and increasing a likelihood of transmissibility.

A second example of an individual unwilfully exposing other individuals to a viral pathogen such as Covid19, would be illustrated as a parental figure who has been tested as positive for Covid19 and has a child who is experiencing a life-threatening emergency such as a burn or an electrocution. As a parent instinctually desires to remain at the side of a child who is experiencing a life-threatening emergency, even when such parent understands that a transmission from such parent to the child is likely to occur when such parent comes into radial proximity of the child, a parent may, in the emergency of a child's condition and circumstance, willingly, though regretfully, come into proximity of a child and attend to a child's immediate interests, as the parental figure may elect that an absence of the parental figure in relation to a child in an emergency circumstance may lead to the immediate mortality of the child, while an exposure to a viral pathogen may as well lead to a mortality of a child, the parental figure's choice to remain in proximity to a child in distress would be based upon a heart-rendering set of choices, though the parental figure may choose to remain in proximity, at a child side, during an emergency as described in the foregoing illustration, as the parental figure's choice is based on a time-sensitive condition of a child in an emergency condition, and an exposure to a viral pathogen as a result of a transmission from a parental figure affords a time to address an exposure from the parental figure's child to a viral pathogen, such as Covid19.

In an effort to provide a concrete working example of a governmental agency emergency mandates, following a directive from the Center for Disease Control (CDC), a utilization of a PPE a following example pertaining to the State of Florida, located in the USA, where Mr. Rick DeSantis presides as Governor is herein provided. Mayors of Cities in Southern Florida where a ‘spike’ in Covid19 cases (were-is), occurring and as well such Mayors recognition that hospitalization (were-are), trending upward in Miami-Dade County, as The State of Florida surpassed a numerical milestone for Covid19 cases and by reporting 100,000 confirmed cases of Covid19 on 22 Monday 2020, a plurality of Southern Florida Mayors recognized a need to get “tougher” with mask mandates by requiring persons to wear face coverings in public setting as Southern Florida (was-is) becoming an epicenter for Covid19 transmission. City of Miami Mayor Francis Suarez was quoted in stating that the City of Miami would be implementing a mask-in-public rule, where everyone will have to be wearing a mask in public. Persons detected to be noncompliant with such mandate face at least one (1) monetary fine. The Southern Florida cities participating in the mask mandate include: Miami, North Miami Beach, Hialeah, Miami Gardens, Aventura, Key Biscayne, Biscayne Park, Pinecrest, West Miami, Miami Shores and El Portal and such mandate as per local Mayor's directive may extend to other cities. Additionally, The State of Florida reported record-setting totals of new cases on the subsequent Thursday, Friday and Saturday, as South Florida remained an epicenter for Covid19 as Miami-Dade County, in specific, (was-is) accountable for 26,239 of The State of Florida's 100,217 confirmed cases as of 22 Monday 2020, illustrating a real ‘spike’ and a real ‘trajectory’ in the confirmed cases of Covid19. The economies of such cities were already in a “shutdown” mode and looking forward to a possible, “re-opening” however, in view of the concept that no medical service, such as the medical service of the present invention (was-is), available to materially mitigate the transmission of Covid19 a likelihood of a partial or full re-opening of the cities economies became jeopardize.

While a best practice of the present invention would be understood as utilizing a poly-metric approach to prevention of contracting a viral pathogen such as Covid19, and practice social distancing and proper hygiene such as hand washing, a false distinction of the present invention would be to understand it as freely licensing an individual to be socially interactive. Governing agencies, such as though not limited to a municipality, a city, a county, a state or a country at a Federal level, often mandate citizen's to practice social distancing and wear Personal Protective Equipment (PPE), when an individual is in a public setting as illustrating in the foregoing description. Within the same light, the processes of the present invention's medical service would afford a mitigation of a viral transmission in a public setting when a government agency, such as though not limited to the foregoing set of governing agencies mandate a utilization of such processes. While persons are compliant and non-compliant with government mandates there is no reason to think persons would materially change their positions should the processes of the present invention be mandated by a government agency. That is to say, select persons will remain compliant and others will remain non-compliant. It should however be understood that an ambition of the present invention is to capitalize on and exploit technological gains occurring in future practice of the technological arts. And, as such equipment as PPE, inclusive of though not limited to face-masks, face-shields, medical grade gloves and hand-sanitizer are often limited or not available to persons in the medical arts and the public at large during a viral pandemic, it should also be understood that most persons of a population, namely, the persons incorporating the population of The United States of America (USA), already possess an electronic device such as though not limited to a cell or smartphone, a wearable device or mobile computer tablet all of which have a means to connect to a network such as the World Wide Web. Consequently, acquiring the processes of the present invention would require downloading and activating an App upon completion of a medical examination by a Medical Professional of an IHS as defined in the present invention in order to actualize the processes of the present invention's medical service the IHS's electronic device.

As the current and expanding epidemic of Covid19 is not only causing, sickness and mortality and as well causing great financial hardship in view of persons not being able to go to work for concern of contracting Covid19, a slowing of the federal economy and associated equities market's devaluation that effects the investments of millions of citizens of the USA, a reasonable course of action by a federal governing agency that is attempting mitigate a viral pandemic and as well stem financial hardships and losses would be to afford a citizenry of its population a means to acquire an electronic device, such as though not limited to a dedicated wearable electronic device engineered to be worn on a wrist of a person and an internet service provider (ISP), so the electronic device can connect to the World Wide Web and hence, provide means for a person who was otherwise financially dispositioned to privately own an electronic device and internet connectivity. As such, a purchasing manager of a government agency may acquire a plurality of electronic devices, such as though not limited to a dedicated wearable wristband electronic device and an associated plurality of ISP connections from a manufacturer or vendor and through a government program, distribute the electronic devices and associated ISPs to persons who meet an economic criteria. In doing so, and while such program would come as an expense to a government agency, a largely offsetting benefit would be the retention of good health of its citizenry, a lowering of mortality rates and general stability of equity markets, all of which would come about through the practice of the present invention. And, while affording its citizenry electronic devices and ISPs is at an expense to a government, such expense would be considered as immaterial and ethically irresponsible to take into accounting balance and justification, notably when the associated costs of providing such program would be materially lower than a financial impact of a viral pandemic such as Covid19.

While a public dialogue in the form of debate may supervene in light of the present invention's interest to provide for a well-being of a population in times of a declared viral pandemic such as the present viral pandemic of Covid19 as to a right of person to protect oneself from a transmission of a viral pathogen, and a compromise of right of a person's medical condition to remain as private. The Inventor of the present invention's reflection upon the processes of the present invention in terms of a right of an individual to protect oneself and a right an individual's health status to remain as private, though individual to the Inventor of the present invention, maintains that the health and benefit of protecting oneself from acquiring and potentially spreading a viral pathogen, such as though not limited to Covid19, far outweigh a compromise of a medical privacy of an individual, notably as the processes of the present invention “Do Not” disclose a personally identifying information about a person with a viral pathogen other than a health condition of such person and such person's proximity to other persons. The Inventor of the present invention therefore defers the adjudication of the balance of such dialogue to a populace of persons where the processes of the present invention are to be practiced. It should be understood and appreciated that a social contact and a social distance practice are already in place in a population, though the processes of the present invention technologically modernize such process, such as current practice of social distancing, that necessarily compels persons of a populace to practice a social distancing from all persons encountered in a social setting, while the processes of the present invention, and, through such technological modernization, provide a means to filter persons who are and who are not in a status of good health. Consequently, a benefit of the present invention is that persons of a populace would not be required to practice a cessation of all social interaction and carry along with their general social affairs in a general reasonable manner.

A medically material pre-condition of practicing the processes of the present invention's medical service would be stipulated in a written and signed contract that is consistent with a law and a right, by an individual and a medical professional, that a partition of a Medical Professional's URL of an individual's medical records remain private and secured, and a partition of a Medical Professional's URL of an individual's medical records be made public, and that an individual, such as IHS ABC123, of the present invention has been tested by a medical professional for a viral pathogen, such as though not limited to Covid19, and provided an IHS number, such as though not limited to, ABC123, and a coordinating MEDIHS number, and where applicable, a government issued MEDIHS, such as though not limited to both the IHS and the MEDIHS of the IHS with a number such as though not limited to ABC123, and a calendar scheduled biometric update, such as a core body temperature check, that could potentially be incorporated into a function of an individual's electronic device, upon a given unit of time as determined by a Medical Professional.

### BACKGROUND OF THE INVENTION IN RELATION TO CURRENT PRACTICE

With relation to the processes of the present invention a current practice in its field of endeavor, there is a failure of all current art taken singularly or in combination to exhibit the inventive leap to a level and degree as the facility of the present invention provides in the present specification, explicitly, its facility of providing an unified a registry and a platform medical service for advising a primary individual of secondary individuals in a radial proximity to the primary individual of a health status of a secondary individual within a radial proximity of the primary individual. More particularly, the present invention provides a unified process for transmissible viral pathogens radial proximity real-time virtual global positioning plotting and social distancing computerized advanced advisory registry driven platform medical service in order to provide a map plot on a MEDIHS of a primary IHS so a primary IHS would know a health status of a secondary IHS with a radial proximity to a primary IHS and follow a protocol of an “Advanced Advisory” on a MEDIHS of primary IHS and take preventative measure as required in order to mitigate a transmission of viral pathogen such as though not limited to Covid19, as “Prevention is the best cure”. As such, no current singular practice of art is efficiently suitable for reference in terms of the inventive leap of the present invention. With these respects, all currently practiced art fails in reference to the conceptual considerations of the present invention.

For clarity purposes, one ordinarily skilled in the art of endeavor to which the present invention pertains, would find an ability to make and practice the present invention as its Specification provides clear and comprehensive recitations of its functioning embodiments along with accurate explanations both in its text and drawings. Upon review of the present Specification, one ordinarily skilled in such art would contemplate the facility of its inventive leap in a manner consistent with ordinary thought patterns as the Specification contains subject matter, disclosed herein, that is described in such a way as to reasonably convey the conceptual qualities of the present invention as the Applicant has demonstrated through the present application by illustrating comprehensively and with detail the process of the present invention and its associated embodiments.

Although the present invention is comprised of a plurality of embodiments inclusive of though not limited to, i.e., a central computer, a network, a securing of the central computer, a CCNTCRM, a MEDIHS of a primary IHS, a coordination of a MEDIHS unique code with unique code of a primary IHS, a MEDIHS of a secondary IHS, an App on a MEDIHS of an primary and secondary IHS, a computerized device of a Medical Professional, a securing of a Medical Professional's URL, a health status of an individual as good as determined by a health professional, a health status of an individual as not good as determined by a health professional, a medical registry, a partitioning of a medical registry as private, a portioning of a medical registry as public and a contract, at the present invention's most fundamental level, the operability of the present invention's processes are largely based upon programming a computer, such as the central computer of the present invention to calculate a data and provide a MEDIHS of a primary and secondary IHS and operational quality of providing an iconic representation of a primary IHS and a secondary IHS that illustrate an IHS in a radial proximity to a primary IHS. It should thereof be recognized that while the embodiments and their manner of operability practiced in the present invention's field of endeavor that no presently practiced art provides an operational facility such as advancing an advisory to a primary MEDIHS of a primary IHS so a primary IHS may know an IHS of a secondary IHS and a radial distance and geographic position of a secondary IHS so a primary IHS may be advised as to what course of action to follow predicated on an information on a primary's MEDIHS data as calculated and transmitted through a network to a MEDIHS of a primary IHS.

As such, and taken along with the concise and comprehensive descriptions in the Specification of the original disclosure as filed, one of ordinary skill in the art to which the present invention pertains would appreciate the present invention's embodiments would not need to perform undue experimentation, as the functioning utilities of the present invention's embodiments are well detailed in the present inventions claims, specification, drawings and abstract. Explanations of the recited processes of the present are in-depth, sound and concise and exhibited with such exacting terms that they grant one ordinarily skilled in the art to which the present invention pertains ample knowledge to comprehend the ingenuity of the inventive leap of the present invention and carry out a building and operation of the present invention. Accordingly, and as the actuation and operation of the present invention is established principally by how the central computer is programmed to govern the embodiments of the present invention, one ordinarily skilled in the art of endeavor to which the present invention pertains would know how to make and practice the processes of the present invention. Additionally, a patron of the present invention would recognize the social facility of the present invention's to provide a map plot of persons within a radial proximity to an IHS and consequently feel more at ease when desiring and/or needing to participate in a social setting such as a work setting.

In reference to the foregoing considerations, it should be recognized that an ambition of the present invention is to provide a population of person with a technologically modernized facility in terms of an advanced advisory on a computerized device in so, life for an individual and a population may proceed in a more normal manner when an organization, such as the World Health Organization, declares a global emergency pertaining to a viral pandemic, such as a recent declaration by the WHO of the viral pathogen of Covid19.

### Steps for How the Central Computer (1) Programmatically Performs a Calculation in Order to Provide a Primary Individual with a Data in Relation to a Real-Time Radial Distance Map Plotting of a Secondary Individual

In light of the present invention's inventive leap of being squarely centered in the conceptual consideration of an operational facility of providing a primary individual with a data in relation to a real-time radial distance map plotting of a secondary individual and a programming of the central computer that effectually provides means to perform such an operational facility, the programming of the central computer is consequently considered as core to the utilities of the processes of the present invention at large. Accordingly, brief and detailed descriptions of the steps of how the central computer is programmed to perform such a function are herein supplied in order for one ordinarily skilled in the art of computer programming to understand how the inventor intends for the desired result providing a primary individual with a data in relation to a real-time radial distance map plotting of a secondary individual is achieved. Steps for programming of the central computer in a method as specified in the current illustration may apply to all programming methods of the central computer concerning the functioning embodiments of the present invention and their respective calculations by the central computer. Further, there are five (5) steps in programming a computer, such as the central computer of the present invention.

### Brief and Detail Descriptions of Programming the Central Computer 1 with Five (5) Steps

With reference to the present invention, a “Brief” description of programming a computer to perform the functions of the five (5) steps may be exercised as:
1) Defining the problem, “Programming a computer provide a primary individual with a data in relation to a real-time radial distance map plotting of a secondary individual”.
2) Planning the solution, “Assigning naming conventions to embodiments and functions and declaring causal order such as chronological”.
3) Coding the program, “Utilization of a program tailored for performing calculations from data”.
4) Static and dynamic testing of the program, “In simulation, prototype and/or practice on at least one (1) occasion”.
5) Documenting the static and dynamic program tests, “Documenting the portions of the program that functioned efficiently or not efficiently in simulation, prototype and/or practice, and returning as required to step four (4) and “re-testing” the program in simulation, prototype and/or practice on at least one (1) occasion to debug the program and maximize its calculative efficiency”.

### Detailed Description of Programming the Central Computer 1 with Five (5) Steps

With reference to the present invention, a ‘Detailed’ description of programming a computer to perform the functions of the five (5) steps may be exercised as:
1) Defining the problem; The problem being defined as, “Programming a computer to provide a primary individual with a data in relation to a real-time radial distance map plotting of a secondary individual”. Providing a primary individual with a data in relation to a real-time radial distance map plotting of a secondary individual may be further defined as; the processes by which the central computer computes a calculation of a real-time GPS data of a primary IHS and a secondary IHS and determines a radial distance between a primary IHS and a secondary and a facility of transmitting the data through the network to a MEDIHS of an primary IHS and to a MEDIHS of a secondary IHS.
2) Planning the solution, Planning the solution being defined as, “Assigning naming conventions to embodiments and functions and declaring causal order such as chronological”. Examples of assigning naming conventions to embodiments and functions of the present invention may include although are not limited to; “Mobile Electronic Device”, as, “Mobile Electronic Device”. Thereafter, declare causal order, such as chronological, in terms of when the functioning embodiments of the present invention, over a duration of time are actualized, inclusive of a succession of the actualization of the embodiments of the present invention over a duration of time, such as the duration of time of a continuous stream of may be further detailed as, though not limited to such causal order as illustrated herein, in which the causal order of the processes of the present invention are actualized. For example, a first process may include although is not limited to, calculating a GPS data of a primary IHS as sent through the network by the primary IHS's MEDIHS. A second process may include a calculating a GPS data of a secondary IHS as sent through the network by the secondary IHS's MEDIHS. A third process may include a processing of a data of a radial distance between a primary and secondary IHS utilizing a GPS data of a primary and secondary IHS. A fourth process may include transmitting a directive such as an advanced advisory to a MEDIHS of a primary and a secondary IHS.
3) Coding the program, “Utilization of a program tailored for performing calculations from a data”. First, select a software program tailored for programming a computer that performs computational calculations from data, such as though not limited to; Fortran™, Python™, CPython™, or C++™. The exemplar program is “Python™”. Next, if the program is already installed on the computer, click “Start”. If the program is not installed, install the program, and click “Start”. After starting the program begin to execute a file script in “IDLE”, by pressing the F5 key on the keyboard on select operating systems. Select “file”>“new file”, a Python™ editor file window will open. Name and save the file on a directory of the Central computer's 1 directory, for example, the local “C” drive with the Python™ extension “.py” as, “Radial Distance.py”. Next, write and save the “Radial Distance.py” program so it is prepared to initialize and perform its function.

Software programming methods and techniques may vary considerably between programs and individual code script writing formats. While there are programming guidelines, as programming is an art, there are also liberties. As such, the enclosed explanation of how to program a computer to perform computational calculations should be considered exemplar. Although the utilization of the exemplar program is Python™, it should not be considered as exclusive, as any software writing program of effective facility could be utilized to write such a program. While the illustration of the exemplar program is sufficient in detail for one ordinarily skilled in the art of computer programming to understand how to make and practice the functioning facility of the present invention in terms of programming the central computer to perform a calculation as the inventor intends the central computer to be programmed, the exemplar illustration should however not be considered as comprehensive.
4) Static and dynamic testing of the program, “In simulation, prototype and/or practice on at least one (1) occasion. A static and/or dynamic program test in simulation, prototype and/or practice may include though is not limited to, static and dynamic and/or testing the program for; A) Basic functionality testing that assures each hardware input keystroke corresponds to the proper software function, crash testing the software program, by trying out all the different combinations of clicks and characters, or edge conditions and restoring code where necessitated. B) Peer code review in order to ensure quality of coding. C) Static code analysis that analyzes the computer software as performed without real working execution of the programs. D) Dynamic testing analysis, which is analysis performed on programs while they are executing in simulation, prototype and/or real working practice. Dynamic testing may be performed by an automated tool, with manual analysis being conducted as program understanding, program comprehension, or code review that enables software ‘inspections and walk-throughs’. E) Utilize a source code tool(s) to perform an analysis on the source code such as; Visual Expert™, Code Compare™, Behavioral Code Analysis™, Fortify Static Code Analyzer™, and/or Veracode™. Any source code automated tool of effective facility could be utilized to perform analysis on source code or bytecode without executing the source code and scan it for weaknesses, such as security vulnerabilities and potential concurrency matters, detects coordination bottlenecks, simulates upcoming knowledge loss used for off-boarding, prioritize re-factorings and technical debt, mobile application behavioral analysis and software composition analysis, reverse-engineering, performs software maintenance, modernization or optimization, calculates software metrics and source lines of code and finds unused objects and methods, identification of duplicate objects, controlling naming conventions, and enforces coding criterions that are configured to run automatically as a capacity of the software build. F) Unit testing to ensure each unit of code is working as expected in static and dynamic testing in simulation, prototype and/or real working practice throughout a series of valid and invalid inputs. G) Single-user performance testing that runs single-user tests at the initial stage of testing that allows examination of the overall performance of load testing to confirm that the test can run successfully with a designated load profile.
5) Documenting the static and dynamic program tests, “Documenting the portions of the program that functioned efficiently or not efficiently and returning as required to step four (4), re-testing the program in simulation, prototype and/or practice on at least one (1) occasion to debug the program”. Debugging the program may include though is not limited to; adding, subtracting or re-assigning naming conventions, adjusting for operational efficiency such as; network latency in order to perform a calculation that incorporates coding adjustments pertaining to network latency in order to determine an accurate calculation of a primary IHS and secondary IHS's global position, adjustments for operational efficiency in various meteorological conditions, and adjustments for operational efficiency for responsiveness to an IHS's inputs in order to program the central computer in its entirety for maximized operational calculative efficiency.

### BRIEF SUMMARY OF THE INVENTION

In essence, the substance of the present invention's conceptual consideration is centered upon a programmatic performance of all of it processes, inclusive of its embodiments, as being operationally unified. The present invention's inventive leap advantages itself above currently practiced art in the field of endeavor in the technological arts of providing a means to mitigate a transmission of a viral pathogen, such as though not limited to, Covid19. The present invention does so by providing a registry and a platform medical service for advancing an advisory to a primary individual of secondary individuals in a radial proximity to the primary individual of a health status of a secondary individual within a radial proximity of the primary individual. More particularly, the present invention provides a unified process for transmissible viral pathogens radial proximity real-time virtual global positioning plotting and social distancing computerized advanced advisory registry driven platform medical service.

As mitigating a viral transmission of a viral pathogen is indispensable as a time-critical exercise of a population of persons, a means to be “advanced an advisory” in the event persons of a population are engaged in a social setting, a remaining “out in front” of a viral transmission is as well critical to the practice of mitigating a viral transmission. The inventive leap and unified processes of the present invention are drawn toward such interests within its field of endeavor of in the technological arts of providing a means to mitigate a transmission of a viral pathogen, such as though not limited to, Covid19 as its processes are comprehensively tailored within all of the respects of its processes and embodiments to provide a platform medical service to a population of person in order to materially mitigate a transmission of a viral pathogen.

### BRIEF DESCRIPTION OF DRAWINGS

FIG. 1 illustrates a central computer containing a CCNTCRM that is connected to a network and receiving, processing and transmitting a data. The central computer is receiving a data sent through a network by a medical professional and processing and equipping the Central Computer with a Non-Transitory Computer Readable Medium, CCNTCRM and the central computer having network connectivity means; processing an assigning an Individual with a Health Status IHS, such as “Good” or “Not good” as determined by a medical professional and encoding the CCNTCRM with a uniquely identifying criteria registry database of an IHS and encoding the CCNTCRM with a uniquely identifying criteria registry database of an Individual's Health Status IHS; processing an outfitting an IHS with a mobile electronic device, MEDIHS, the MEDIHS being equipped with a network connectivity, a global positioning system, a compass, a computerized advisory application means, a keyboard and a monitor; processing an determining an IHS as primary and determining an IHS as secondary and determining a MEDIHS as primary and a MEDIHS as secondary; processing a determining a MEDIHS as independent or dedicated, assigning an IHS and an MEDIHS with unique identifying codes and coordinating an IHS and an MEDIHS unique identifying codes; processing a programming the central computer to perform a global positioning plotting of one or more MEDIHS; processing an encoding the CCNTCRM with a registry database; processing an encoding the CCNTCRM registry database for a purpose of recording a health status of an individual; processing a providing a Medical Professional a means to record an IHS into a registry database of the CCNTCRM by utilization of a computerized device and a network; processing a programming the central computer to process a global positioning data; processing an interconnecting a MEDIHS with the central computer utilizing the MEDIHS's network connectivity means and central computer's network connectivity means; processing a programming a MEDIHS to display a global positioning plotting of one or more other MEDIHS on a monitor of a MEDIHS; processing a programming the central computer to transfer a global positing data of a plurality of MEDIHS through the network to a plurality of MEDIHS; processing a programming the central computer to transfer an advisory through the network to one or a plurality of MEDIHS; processing a programing the central computer to calculate a IHS as determined by a medical professional and the IHS being transferred through the network to a MEDIHS and being displayed on the monitor of the MEDIHS; processing a programming the central computer to determine a global position of a primary and secondary MEDIHS and “only” when a radial distance of a primary MEDIHS is being breached by a secondary MEDIHS with a health status as “Not good” the central computer programmatically transmits an “Advanced Advisory” to a primary and secondary MEDIHS of primary IHS and secondary IHS in real time; processing a providing an instruction to an IHS to follow a protocol when an advanced advisory is received on a MEDIHS of an IHS; processing a determining an IHS by a health professional as, “in good health” or, “not in good health” and programming the central computer to recognize a health status of an IHS as determined by a health professional as, “in good health” or, “not in good health”; processing a providing a computerized device of a Medical Professional a means to input a IHS data into the computerized device of the Medical Professional, the computerized device of the Medical Professional having network connectivity means, connecting the computerized device of the Medical Professional to the network, and the Medical Professional transmitting a IHS data through the network to the central computer and the central computer storing a IHS data in the CCNTCRM; processing a partitioning and securing a registry database on the central computer and securing a URL of a Medical Professional; processing a drafting a medical contract with a term and a condition that is consistent with a law and a right and providing the contract with a signatory and dating line; programming a encoding a registry database of the central computer with a plurality of IHS's in a manner consistent with a law and a right as expressed in a term and a condition of the contract; and processing a programmatically performing all processes as unified.

FIG. 2 illustrates a primary IHS wearing a PPE in the form of a face mask and a MEDIHS of the IHS and a network connectivity means of the MEDIHS.

FIG. 3 illustrates a primary IHS wearing a PPE in the form of a face mask and an alternate embodiment of an MEDIHS of the primary IHS in the form of a wearable wrist computerized device and a network connectivity means of the MEDIHS.

FIG. 4 illustrates a secondary IHS wearing a PPE in the form of a face mask and a MEDIHS of the secondary IHS and a network connectivity means of the MEDIHS.

FIG. 5 illustrates a secondary IHS and a MEDIHS of the secondary IHS and a network connectivity means of the MEDIHS and a viral pathogen of the IHS.

FIG. 6 illustrates a Medical Professional wearing a PPE in the form of a face mask.

FIG. 7 illustrates a Medical Professional wearing a PPE a primary IHS wearing a PPE in the form of a face mask, the Medical Professional wearing an alternate embodiment of a MEDIHS in the form a wearable wrist computerized device and an network connectivity means of the Medical Professional's MEDIHS, a medical equipment in the form of a cotton swab, a medical equipment in the form a pathogen test kit, a primary IHS with a health status as “Good”, an icon representing the primary IHS, a MEDIHS of the primary IHS and a network connectivity of the primary IHS's MEDIHS.

FIG. 8 illustrates a Medical Professional wearing a PPE a primary IHS wearing a PPE in the form of a face mask, the Medical Professional wearing an alternate embodiment of a MEDIHS in the form a wearable wrist computerized device and an network connectivity means of the Medical Professional's MEDIHS, a medical equipment in the form of a cotton swab, a medical equipment in the form a viral pathogen test kit, a PPE in the form of a face mask of a secondary IHS, a secondary IHS with a health status as “Good”, a MEDIHS of the secondary IHS and a network connectivity of the secondary IHS's MEDIHS.

FIG. 9 illustrates a Medical Professional wearing a PPE a primary IHS wearing a PPE in the form of a face mask, the Medical Professional wearing an alternate embodiment of a MEDIHS in the form a wearable wrist computerized device and an network connectivity means of the Medical Professional's MEDIHS, a medical equipment in the form of a cotton swab, a medical equipment in the form a viral pathogen test kit, a PPE in the form of a face mask of a secondary IHS, a secondary IHS with a health status as “Not good”, a MEDIHS of the secondary IHS and a network connectivity of the secondary IHS's MEDIHS.

FIG. 10 illustrates a Medical Professional wearing a PPE in the form of a face mask and inputting a health data of a primary IHS (ABC123), into a health registry on a computerized device of the Medical Professional in the form of the primary IHS's (ABC123), MEDIHS unique code (ABC123), the primary IHS's (ABC123), unique code, a security icon of the URL of the Medical Professional, a privately secured partition of data of the health registry with a health status of a secondary IHS (ABC123) as “Good”, a public access partition of the health registry, a network connectivity means of the Medical Professional's computerized device and an icon of a health status of a primary IHS (ABC123).

FIG. 11 illustrates a Medical Professional wearing a PPE in the form of a face mask and inputting a health data of a secondary IHS (DEF456), into a health registry on a computerized device of the Medical Professional in the form of the secondary IHS's (DEF456) MEDIHS unique code, the secondary IHS's (DEF456) unique code, a security icon of the URL of the Medical Professional, a privately secured partition of data of the health registry, a public access partition of the health registry with a health status of a secondary IHS (DEF456) as “Good”, a network connectivity means of the Medical Professional's computerized device and an icon of a health status of a secondary IHS (DEF456).

FIG. 12 illustrates a Medical Professional wearing a PPE in the form of a face mask and inputting a health data of a secondary IHS (GHI789), into a health registry on a computerized device of the Medical Professional in the form of the secondary IHS's MEDIHS unique code , the secondary IHS's (GHI789) unique code, a security icon of the URL of the Medical Professional, a privately secured partition of data of the health registry, a public access partition of the health registry with a health status of a secondary IHS as “Not good”, a network connectivity means of the Medical Professional's computerized device and an icon of a health status of a secondary IHS (GHI78).

FIG. 13 illustrates a detailed view of a monitor of computerized device of a Medical Professional of a secondary IHS (GHI789), a health registry on a computerized device of the Medical Professional in the form of the secondary IHS's MEDIHS (GHI789) unique code, the secondary IHS's unique code (GHI789), a security icon of the URL of the Medical Professional, a computerized device of the Medical Professional, a privately secured partition data of the health registry, a public access partition of the health registry with a health status of a secondary IHS as “Not good”, an icon representing a health status of an IHS (GHI789), a network connectivity means of the Medical Professional's computerized device and an icon of a health status of a secondary IHS (GHI789).

FIG. 14 illustrates an IHS holding a MEDIHS of the IHS, a network connectivity means of an IHS's MEDIHS, the IHS is engaging an “App” of the present invention on the MEDIHS of the IHS.

FIG. 15 illustrates a primary IHS holding a MEDIHS of the primary IHS, a network connectivity means of a primary IHS's MEDIHS, the MEDIHS of the primary IHS is displaying a map plot of a radial distance map plot of the primary IHS inclusive of an icon of the primary IHS, an icon of a secondary IHS in a radial proximity to the primary IHS with a secondary IHS health status as “Good”, and an icon of a secondary IHS in a radial proximity to the primary IHS with a secondary IHS health status as “Not good”.

FIG. 16 illustrates a detailed view of monitor of a primary IHS's MEDIHS of the primary IHS, the MEDIHS of the primary IHS is displaying a map plot of a radial distance of the primary on its monitor inclusive of an icon of the primary IHS, a secondary IHS with a “Unassigned” value, an icon of secondary IHS in a radial proximity to the primary IHS with a secondary IHS health status as “Good”, and an icon of a secondary IHS in a radial proximity to the primary IHS with a secondary IHS health status as “Not good”, and a compass. Note: a secondary IHS with an unassigned value does not display on a monitor of a primary IHS's MEDIHS and thereof, is illustrated with an intermittent line. The present invention provides a compass means and therefore a primary IHS utilizing the present inventions process on a MEDIHS of a primary IHS can both determine a geographical orientation of secondary IHS that appears on the monitor of the primary MEDIHS of the IHS, such as a secondary IHS with a health status as, “Good” and a secondary IHS with a health status as, Not good” and a geographical orientation of an unassigned individual utilizing a compass embodiment of primary IHS's MEDIHS and visually verifying the orientation of a secondary IHS with a status as unassigned in relation to a geographical position of the primary IHS.

FIG. 17 illustrates a detailed view of a monitor of a primary IHS's MEDIHS displaying an “Advanced Advisory”.

FIG. 18 illustrates a Medical Professional wearing a PPE in the form of a face mask, a primary IHS wearing a PPE in the form of a face mask, a MEDIHS of the primary IHS, a network connectivity means of the MEDIHS, an alternate embodiment of a MEDIHS of a Medical Professional in the form of a wearable wrist computerized device, a network connectivity means of the MEDIHS of the Medical Professional, and a contract with a signatory line.

FIG. 19 illustrates a programmatically performing all processes as unified and a claim number and limitation number of the present invention.

### DETAILED DESCRIPTION OF DRAWINGS

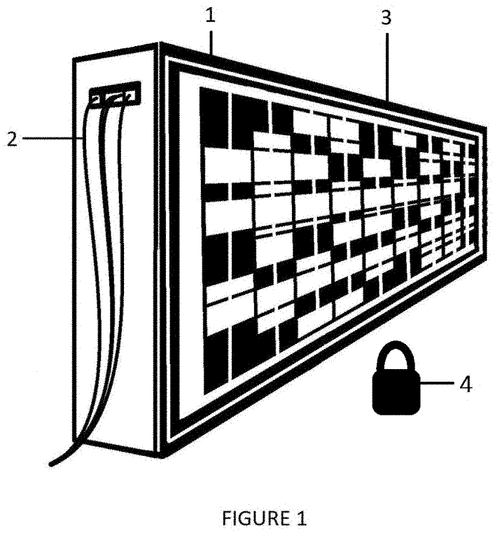

FIG. 1 illustrates a central computer 1 containing a CCNTCRM 3 that is connected to a network 2 and receiving, processing and transmitting a data. The central computer 1 is receiving a data sent through a network 2 by a Medical Professional 12 and processing and equipping the central computer 1 with a Non-Transitory Computer Readable Medium, CCNTCRM 3 and the central computer 1 having network 2 connectivity means 8 and being secured 4; the central computer 1 is processing an assigning an Individual with a Health Status IHS, such as “Good” 15 or “Not good” 18 as determined by a Medical Professional 12 and encoding the CCNTCRM 3 with a uniquely identifying criteria registry database of an IHS and encoding the CCNTCRM 3 with a uniquely identifying criteria registry database of an Individual's Health Status IHS; processing an outfitting an IHS with a mobile electronic device, MEDIHS 7, the MEDIHS 7 being equipped with a network 2 connectivity, a global positioning system, a compass, a computerized advisory application means, a keyboard and a monitor; processing an determining an IHS as primary 5 and determining an IHS as secondary 10 and determining a MEDIHS 7 as primary 5 and a MEDIHS 7 as secondary 10; processing a determining a MEDIHS 7 as independent or dedicated, assigning an IHS and an MEDIHS 7 with unique identifying codes and coordinating an IHS and an MEDIHS 7 unique identifying codes; processing a programming the central computer 1 to perform a global positioning plotting of one 1 or more MEDIHS 7; processing an encoding the CCNTCRM 3 with a registry database; processing an encoding the CCNTCRM 3 registry database for a purpose of recording a health status of an individual; processing a providing a Medical Professional 12 a means to record an IHS into a registry database of the CCNTCRM 3 by utilization of a computerized device and a network 2; processing a programming the central computer 1 to process a global positioning data; processing an interconnecting a MEDIHS 7 with the central computer 1 utilizing the MEDIHS 7's network 2 connectivity means 8 and central computer 1's network 2 connectivity means 8; processing a programming a MEDIHS 7 to display a global positioning plotting of one 1 or more other MEDIHS 7 on a monitor of a MEDIHS 7; processing a programming the central computer 1 to transfer a global positing data of a plurality of MEDIHS 7 through the network 2 to a plurality of MEDIHS 7; processing a programming the central computer 1 to transfer an advisory through the network 2 to one 1 or a plurality of MEDIHS 7; processing a programing the central computer 1 to calculate a IHS as determined by a Medical Professional 12 and the IHS being transferred through the network 2 to a MEDIHS 7 and being displayed on the monitor of the MEDIHS 7; processing a programming the central computer 1 to determine a global position of a primary 5 and secondary 10 MEDIHS 7 and “only” when a radial distance of a primary 5 MEDIHS 7 is being breached by a secondary 10 MEDIHS 7 with a health status as “Not good” 18 the central computer 1 programmatically transmits an “Advanced Advisory” to a primary 5 and secondary 10 MEDIHS 7 of primary 5 IHS and secondary 10 IHS in real time; processing a providing an instruction to an IHS to follow a protocol when an advanced advisory is received on a MEDIHS 7 of an IHS; processing a determining an IHS by a health professional as, “in good health” 15 or, “not in good health” 18 and programming the central computer 1 to recognize a health status of an IHS as determined by a health professional as, “in good health” 15 or, “not in good health” 18; processing a providing a computerized device of a Medical Professional 12 a means to input a IHS data into the computerized device of the Medical Professional 12, the computerized device of the Medical Professional 12 having network 2 connectivity means 8, connecting the computerized device of the Medical Professional 12 to the network 2, and the Medical Professional 12 transmitting a IHS data through the network 2 to the central computer 1 and the central computer 1 storing a IHS data in the CCNTCRM 3; processing a partitioning and securing a registry database on the central computer 1 and securing a URL of a Medical Professional 12; processing a drafting a medical contract with a term and a condition that is consistent with a law and a right and providing the contract with a signatory and dating line; programming a encoding a registry database of the central computer 1 with a plurality of IHS's in a manner consistent with a law and a right as expressed in a term and a condition of the contract; and processing a programmatically performing all processes as unified.

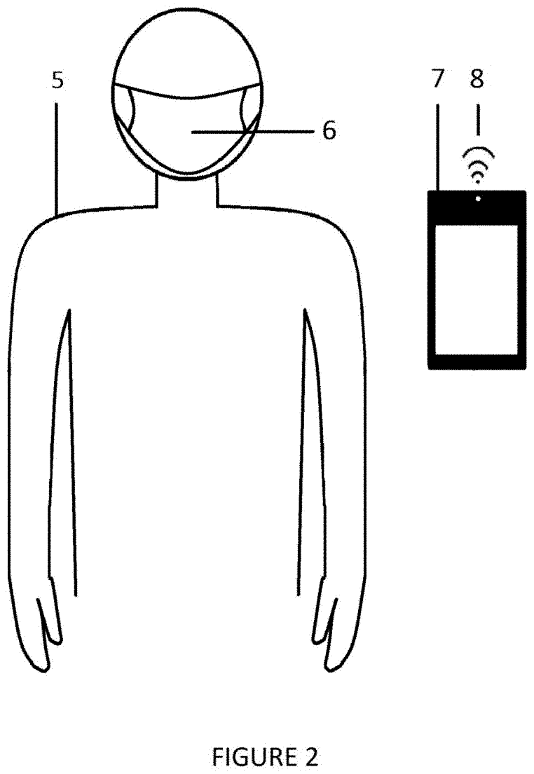

FIG. 2 illustrates a primary 5 IHS wearing a PPE 6 in the form of a face mask and a MEDIHS 7 of the primary IHS and a network 2 connectivity means 8 of the MEDIHS 7.

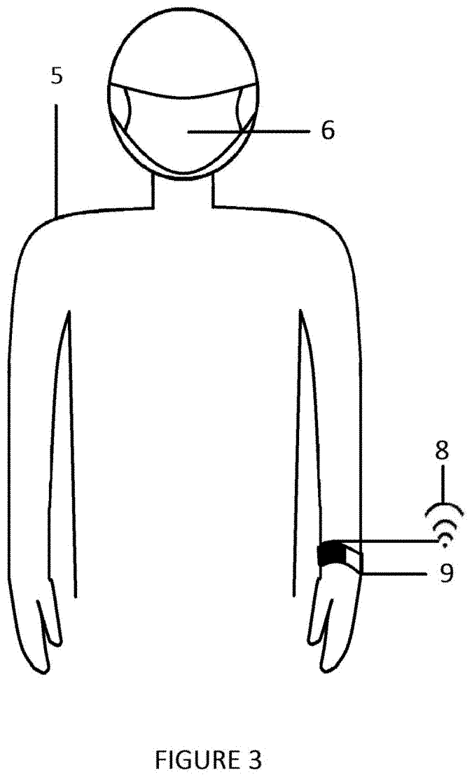

FIG. 3 illustrates a primary 5 IHS wearing a PPE 6 in the form of a face mask and an alternate embodiment 9 of an MEDIHS 7 of the primary 5 IHS in the form of a wearable wrist computerized device and a network 2 connectivity means 8 of the MEDIHS 7.

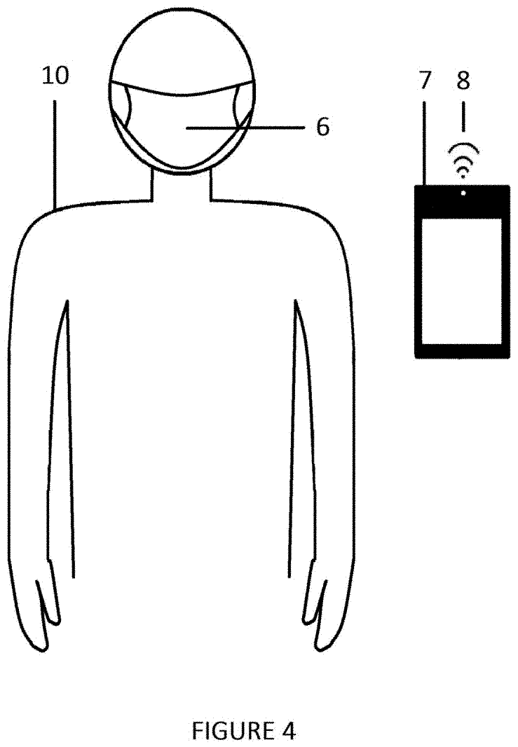

FIG. 4 illustrates a secondary 10 IHS wearing a PPE 6 in the form of a face mask and a MEDIHS 7 of the secondary 10 IHS and a network 2 connectivity means 8 of the MEDIHS 7.

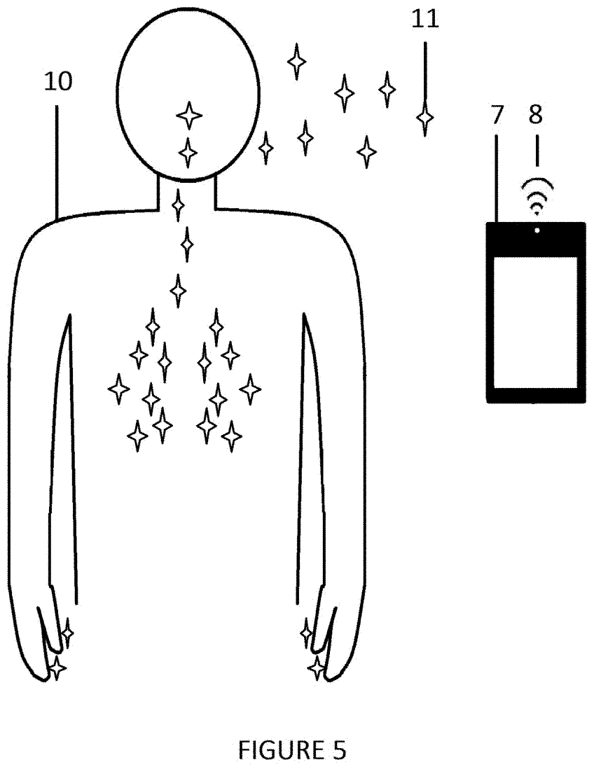

FIG. 5 illustrates a secondary 10 IHS and a MEDIHS 7 of the secondary 10 IHS and a network 2 connectivity means 8 of the MEDIHS 7 and a viral pathogen 11 of the IHS.

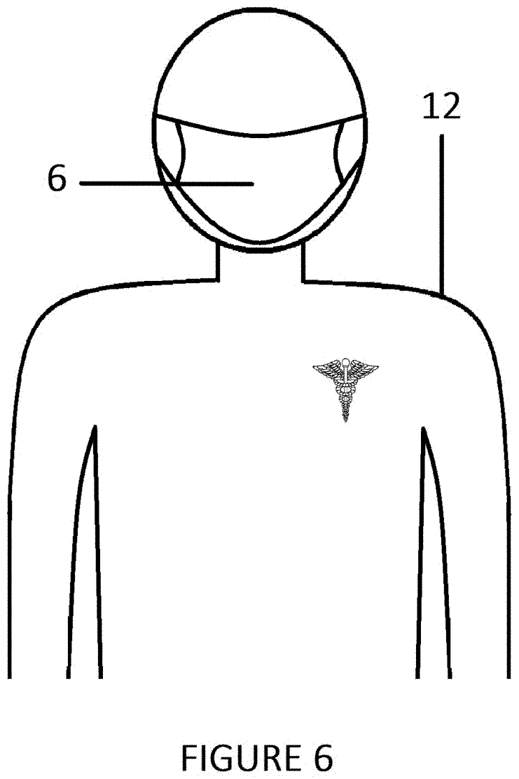

FIG. 6 illustrates a Medical Professional 12 wearing a PPE 6 in the form of a face mask.

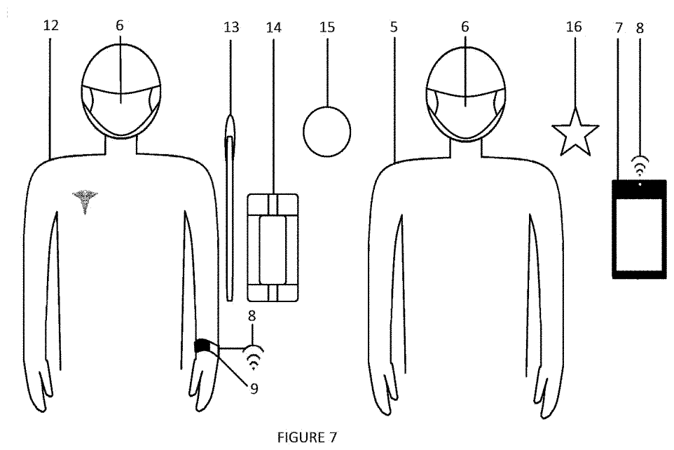

FIG. 7 illustrates a Medical Professional 12 wearing a PPE 6 a primary 5 IHS wearing a PPE 6 in the form of a face mask, the Medical Professional 12 wearing an alternate embodiment 9 of a MEDIHS 7 in the form a wearable wrist computerized device and an network 2 connectivity means 8 of the Medical Professional 12's MEDIHS 7, a medical equipment in the form of a cotton swab 13, a medical equipment in the form a viral pathogen 11 test kit 14, a primary 5 IHS with a health status as “Good” 15, an icon 16 representing the primary 5 IHS, a MEDIHS 7 of the primary 5 IHS and a network 2 connectivity of the primary 5 IHS's MEDIHS 7.

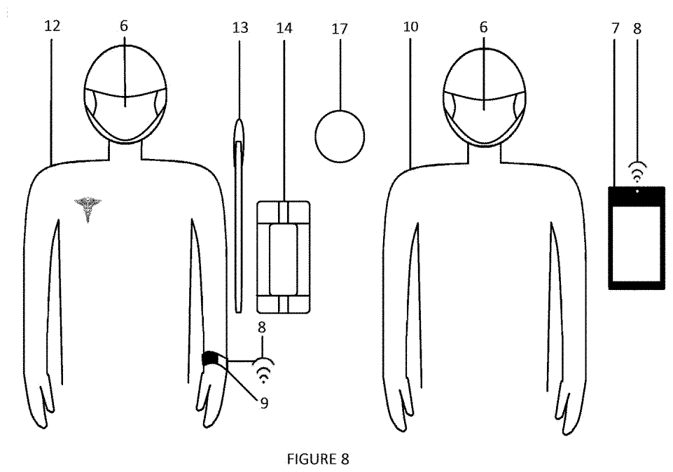

FIG. 8 illustrates a Medical Professional 12 wearing a PPE 6 a primary 5 IHS wearing a PPE 6 in the form of a face mask, the Medical Professional 12 wearing an alternate embodiment 9 of a MEDIHS 7 in the form a wearable wrist computerized device and an network 2 connectivity means 8 of the Medical Professional 12's MEDIHS 7, a medical equipment in the form of a cotton swab 13, a medical equipment in the form a viral pathogen 11 test kit 14, a PPE 6 in the form of a face mask of a secondary 10 IHS, a secondary 10 IHS with a health status as “Good” 17, a MEDIHS 7 of the secondary 10 IHS and a network 2 connectivity of the secondary 10 IHS's MEDIHS 7.

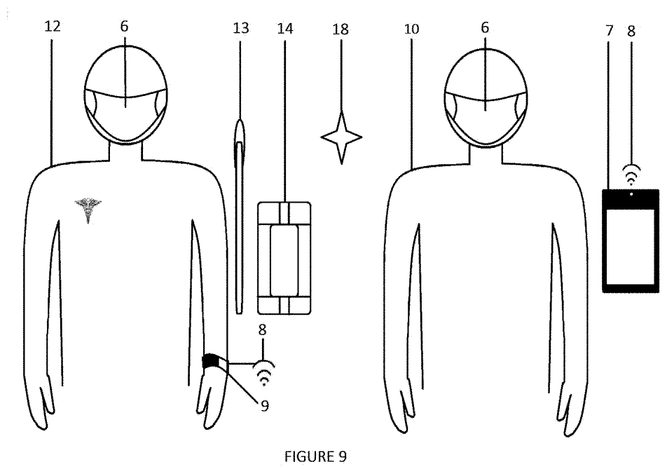

FIG. 9 illustrates a Medical Professional 12 wearing a PPE 6 a primary 5 IHS wearing a PPE 6 in the form of a face mask, the Medical Professional 12 wearing an alternate embodiment 9 of a MEDIHS 7 in the form a wearable wrist computerized device and an network 2 connectivity means 8 of the Medical Professional's 12 MEDIHS 7, a medical equipment in the form of a cotton swab 13, a medical equipment in the form a viral pathogen 11 test kit 14, a PPE 6 in the form of a face mask of a secondary 10 IHS, a secondary 10 IHS with a health status as “Not good” 18, a MEDIHS 7 of the secondary 10 IHS and a network 2 connectivity of the secondary 10 IHS's MEDIHS 7.

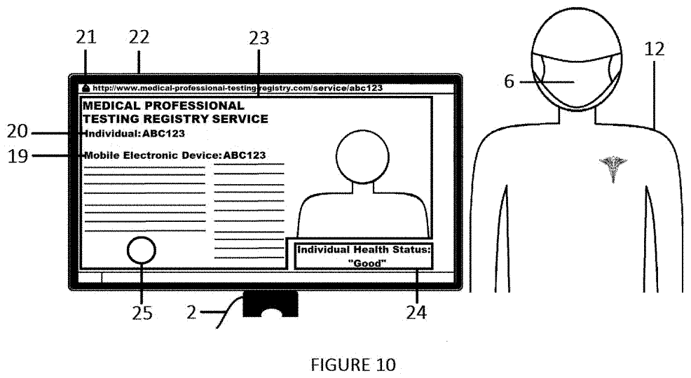

FIG. 10 illustrates a Medical Professional 12 wearing a PPE 6 in the form of a face mask and inputting a health data of a primary 5 IHS (ABC123), into a health registry on a computerized device of the Medical Professional 12 in the form of the primary 5 IHS's (ABC123), MEDIHS 7 unique code (ABC123), 19, the primary 5 IHS's (ABC123), unique code, 20, a security icon of the URL of the Medical Professional 12, a privately secured partition 23 of data of the health registry with a health status of a secondary 10 IHS (ABC123) as “Good” 17, a public access partition 24 of the health registry, a network 2 connectivity means 8 of the Medical Professional 12's computerized device and an icon of a health status of a primary 5 IHS (ABC123).

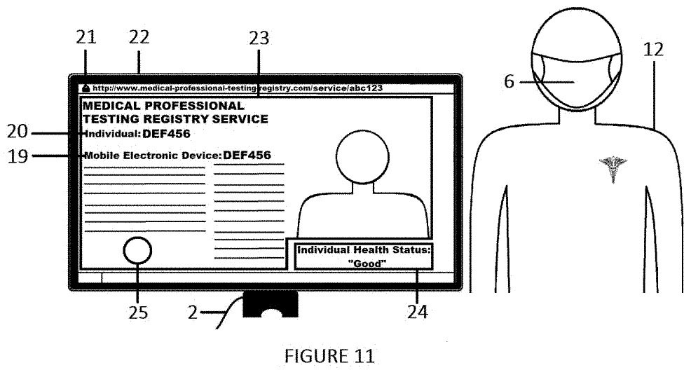

FIG. 11 illustrates a Medical Professional 12 wearing a PPE 6 in the form of a face mask and inputting a health data of a secondary 10 IHS (DEF456), into a health registry on a computerized device of the Medical Professional 12 in the form of the secondary 10 IHS's (DEF456) MEDIHS 7 unique code 19, the secondary 10 IHS's (DEF456) unique code 20, a security icon of the URL of the Medical Professional 12, a privately secured partition 23 of data 19 of the health registry, a public access partition 24 of the health registry with a health status of a secondary 10 IHS (DEF456) as “Good” 17, a network 2 connectivity means 8 of the Medical Professional 12's computerized device and an icon of a health status of a secondary 10 IHS (DEF456).

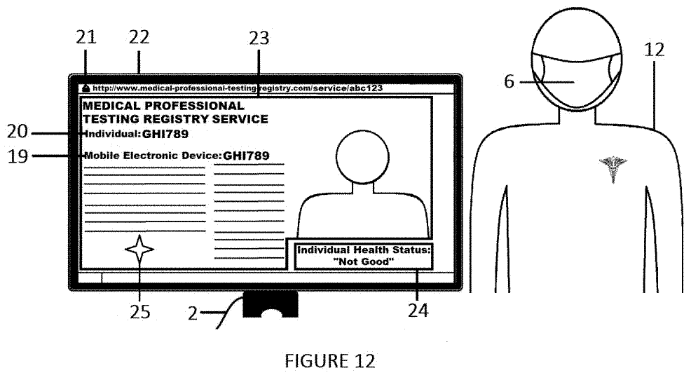

FIG. 12 illustrates a Medical Professional 12 wearing a PPE 6 in the form of a face mask and inputting a health data of a secondary 10 IHS (GHI789), into a health registry on a computerized device of the Medical Professional 12 in the form of the secondary 10 IHS's MEDIHS 7 unique code 19, the secondary 10 IHS's (GHI789) unique code 20, a security icon of the URL of the Medical Professional 12, a privately secured partition 23of data of the health registry, a public access partition 24 of the health registry with a health status of a secondary 10 IHS as “Not good” 18, a network 2 connectivity means 8 of the Medical Professional 12's computerized device and an icon of a health status of a secondary 10 IHS (GHI789).

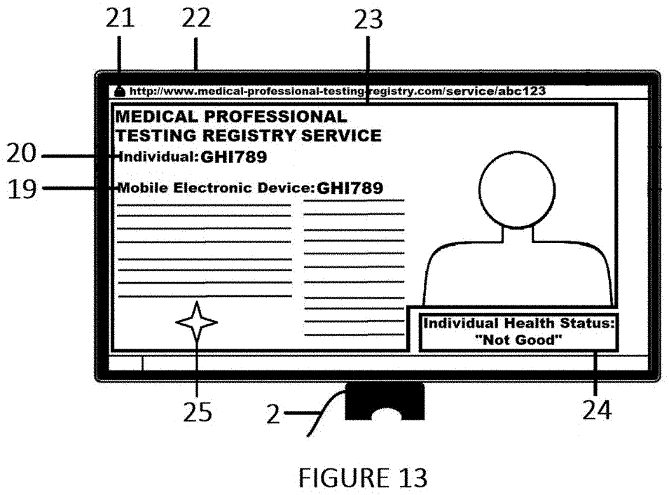

FIG. 13 illustrates a detailed view of a monitor of computerized device of a Medical Professional 12 of a secondary 10 IHS (GHI789), a health registry on a computerized device of the Medical Professional 12 in the form of the secondary 10 IHS's MEDIHS 7 (GHI789) unique code 19, the secondary 10 (GHI789) IHS's unique code 20, a security icon of the URL of the Medical Professional 12, a security icon of the URL of the Medical Professional, 21, a computerized device of the Medical Professional 22 a privately secured partition 23 of data of the health registry, a public access partition 24 of the health registry with a health status of a secondary 10 IHS as “Not good”18, an icon 25 representing a health status of an IHS (GHI789), a network 2 connectivity means 8 of the Medical Professional's 12 computerized device and an icon of a health status of a secondary 10 IHS (GHI789).

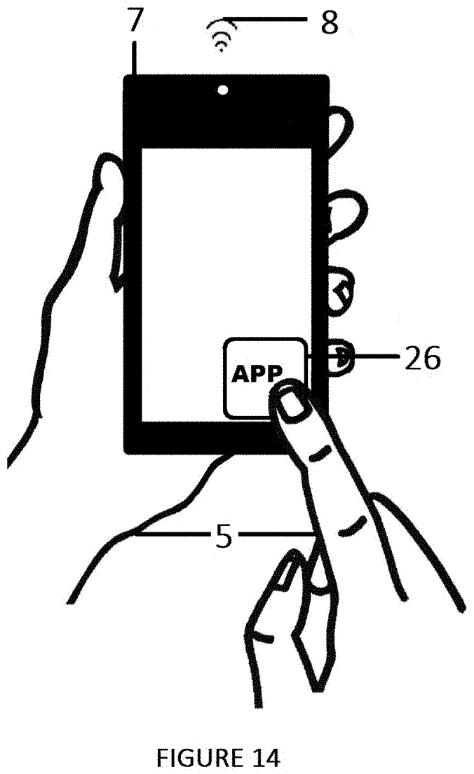

FIG. 14 illustrates an IHS holding a MEDIHS 7 of the IHS, a network 2 connectivity means 8 of an IHS's MEDIHS 7, the IHS is engaging an “App” 26 of the present invention on the MEDIHS 7 of the IHS.

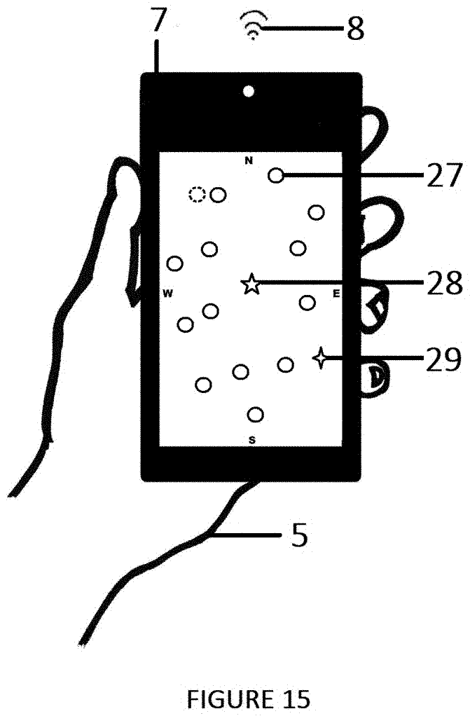

FIG. 15 illustrates a primary 5 IHS holding a MEDIHS 7 of the primary 5 IHS, a network 2 connectivity means 8 of a primary 5 IHS's MEDIHS 7, the MEDIHS 7 of the primary 5 IHS is displaying a map plot of a radial distance map plot of the primary 5 IHS inclusive of an icon 28 of the primary 5 IHS, an icon 27 of a secondary 10 IHS in a radial proximity to the primary IHS with a secondary IHS health status as “Good” 15, and an icon 29 of a secondary 10 IHS in a radial proximity to the primary IHS with a secondary IHS health status as “Not good”18.

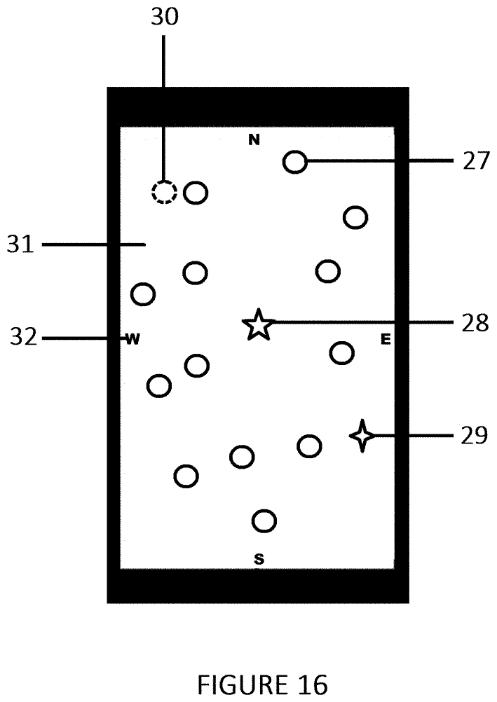

FIG. 16 illustrates a detailed view of monitor of a primary 5 IHS's MEDIHS 7 of the primary 5 IHS, the MEDIHS 7 of the primary 5 IHS is displaying a map plot of a radial distance of the primary 5 on its monitor 31 inclusive of an icon 28 of the primary 5 IHS, a secondary 10 IHS with a “Unassigned” 30 value, Note: a secondary 10 IHS with an unassigned 30 value is not displayed on a monitor 30 of a primary 5 IHS's MEDIHS 7 and thereof, is illustrated with an intermittent line 30, an icon 27 of secondary 10 IHS in a radial proximity to the primary IHS with a secondary 10 IHS health status as “Good” 15, and an icon 29 of a secondary 10 IHS in a radial proximity to the primary 5 IHS with a secondary 10 IHS health status as “Not good” 18, and a compass 32. Note: a secondary 10 IHS with an unassigned 30 value does not display on a monitor of a primary IHS's MEDIHS and thereof, is illustrated with an intermittent line 30. The present invention provides a compass 32 means and therefore a primary 5 IHS utilizing the present inventions process on a MEDIHS 7 of a primary IHS can both determine a geographical orientation of secondary 10 IHS that appears on the monitor 31 of the primary 5 MEDIHS of the IHS, such as a secondary 10 IHS with a health status as, “Good” and a secondary 10 IHS with a health status as, Not good” and a geographical orientation of an unassigned 30 individual utilizing a compass 32 embodiment of primary 5 IHS's MEDIHS 7 and visually verifying the orientation of a secondary 10 IHS with a status as unassigned 30 in relation to a geographical position of the primary 5 IHS.

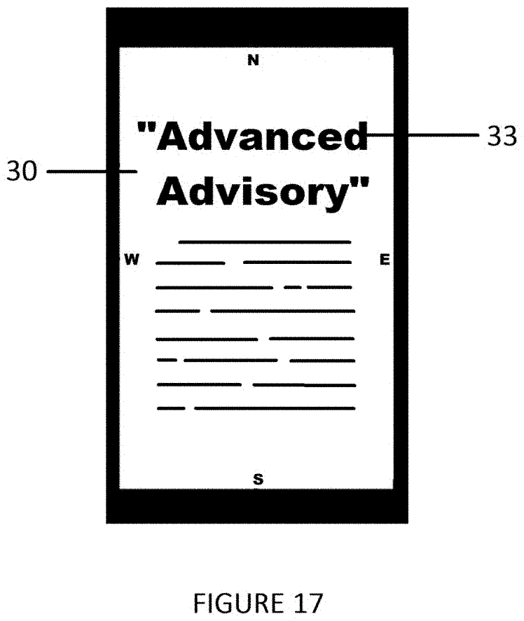

FIG. 17 illustrates a detailed view of a monitor 30 of a primary 5 IHS's MEDIHS 7 displaying an “Advanced Advisory” 33.

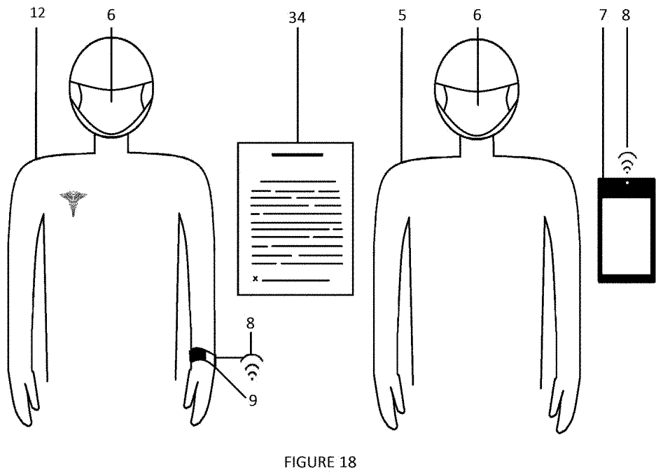

FIG. 18 illustrates a Medical Professional 12 wearing a PPE 6 in the form of a face mask, a primary 5 IHS wearing a PPE 6 in the form of a face mask, a MEDIHS 7 of the primary 5 IHS, a network 2 connectivity means 8 of the MEDIHS 7, an alternate embodiment 9 of a MEDIHS 7 of a Medical Professional 12 in the form of a wearable wrist computerized device, a network 2 connectivity means 8 of the MEDIHS 7 of the Medical Professional 12, and a contract 34 with a signatory line.

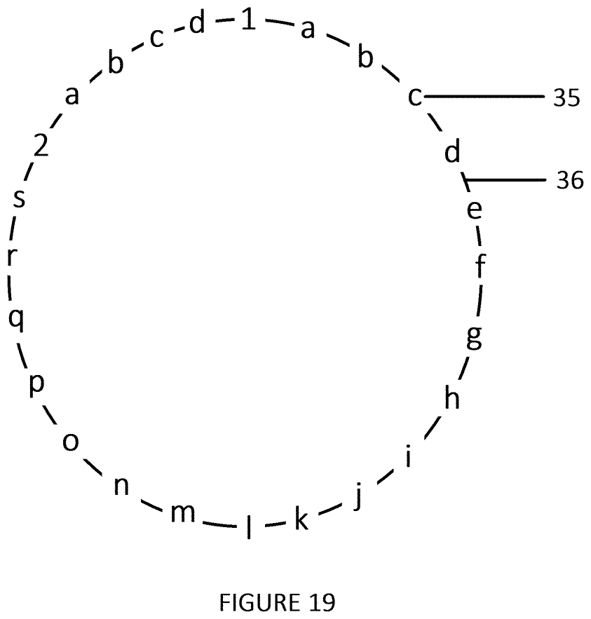

FIG. 19 illustrates a programmatically performing all processes as unified 36 and a claim number and limitation number 35 of the present invention.

### Remarks

The foregoing description conveys the soundest understanding of the objectives, advantages and inventive leap of the present invention. Diverse embodiments may be formed from the inventive concept of the present invention. It is to be understood that matter disclosed herein is to be interpreted not as in a limiting sense, though rather as illustrative. For the purpose of this disclosure, like reference numerals in the figures shall refer to like features unless otherwise indicated or is obvious by context. The subject device and method of use is sometimes referred to as the device, the invention, the process, the technology, the machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation of a condition of a phraseology.

Applicant considers the present application to be in condition for allowance. The present invention claims participation in the United States Patent and Trademark Office's COVID-19 Prioritized Examination Pilot Program for small and micro entities and is inclusive of Doc Code: TRACK1.COVID/Document Description: COVID-19 Prioritized Examination Request, for such interests, the undersigned Inventor of record invites the Examiner to call the undersigned Inventor of record in light of potential objections and/or rejections on behalf of the Examiner in order to clarify potential objections and/or rejections in advance of a disposition of the Examiner other than an allowance at the provided telephone number of the Inventor of record, so the present application may maintain an expedited examination as stipulated in the United States Patent and Trademark Office's COVID-19 Prioritized Examination Pilot Program.

I claim:

1. Unified process for transmissible viral pathogen radial proximity real-time virtual global positioning plotting and social distancing computerized advanced advisory registry driven platform medical service comprising; a central computer, a network, a mobile electronic device, and a computerized application comprised of the steps of;
a) equipping the Central Computer with a Non-Transitory Computer Readable Medium, (CCNTCRM) and the central computer having network connectivity means;
b) assigning an Individual with a Health Status (IHS), such as “Good” or “Not good” as determined by a medical professional and encoding the CCNTCRM with a uniquely identifying criteria registry database of an IHS and encoding the CCNTCRM with a uniquely identifying criteria registry database of an Individual's Health Status (IHS);
c) outfitting an IHS with a mobile electronic device, (MEDIHS), the MEDIHS being equipped with a network connectivity, a global positioning system, a compass, a computerized advisory application means, a keyboard and a monitor;
d) determining an IHS as primary and determining an IHS as secondary and determining a MEDIHS as primary and a MEDIHS as secondary;
e) determining a MEDIHS as independent or dedicated, assigning an IHS and an MEDIHS with unique identifying codes and coordinating an IHS and an MEDIHS unique identifying codes;
f) programming the central computer to perform a global positioning plotting of one (1) or more MEDHIS;
g) encoding the CCNTCRM with a registry database;
h) encoding the CCNTCRM registry database for a purpose of recording a health status of an individual;
i) providing a Medical Professional a means to record an IHS into a registry database of the CCNTCRM by utilization of a computerized device and a network;
j) programming the central computer to process a global positioning data;
k) interconnecting a MEDIHS with the central computer utilizing the MEDHIS's network connectivity means and central computer's network connectivity means;
l) programming a MEDIHS to display a global positioning plotting of one (1) or more other MEDHIS on a monitor of a MEDIHS;
m) programming the central computer to transfer a global positing data of a plurality of MEDIHS through the network to a plurality of MEDIHS;
n) programming the central computer to transfer an advisory through the network to one (1) or a plurality of MEDIHS;
o) programing the central computer to calculate a IHS as determined by a medical professional and the IHS being transferred through the network to a MEDIHS and being displayed on the monitor of the MEDIHS;
p) programming the central computer to determine a global position of a primary and secondary MEDIHS and “only” when a radial distance of a primary MEDIHS is being breached by a secondary MEDIHS with a health status as “Not good” the central computer programmatically transmits an “Advanced Advisory” to a primary and secondary MEDHIS of primary IHS and secondary IHS in real time;
q) providing an instruction to an IHS to follow a protocol when an advanced advisory is received on a MEDIHS of an IHS;
r) determining an IHS by a health professional as, “in good health” or, “not in good health” and programming the central computer to recognize a health status of an IHS as determined by a health professional as, “in good health” or, “not in good health”; and
s) providing a computerized device of a Medical Professional a means to input a IHS data into the computerized device of the Medical Professional, the computerized device of the Medical Professional having network connectivity means, connecting the computerized device of the Medical Professional to the network, and the Medical Professional transmitting a IHS data through the network to the central computer and the central computer storing a IHS data in the CCNTCRM;

2. A process of claim 1 further comprising the steps of:
a) partitioning and securing a registry database on the central computer and securing a URL of a Medical Professional;
b) drafting a medical contract with a term and a condition that is consistent with a law and a right and providing the contract with a signatory and dating line;
c) encoding a registry database of the central computer with a plurality of IHS's in a manner consistent with a law and a right as expressed in a term and a condition of the contract; and
d) programmatically performing all processes as unified.
